(19) United States
(12) Patent Application Publication
(10) Pub. No.: US 2022/0387434 A1
(43) Pub. Date: Dec. 8, 2022

(54) COMBINATION OF A BTK INHIBITOR AND AN MDM2 INHIBITOR FOR CANCER TREATMENT

(71) Applicant: QUOGUE IP HOLDINGS LLC, New York, NY (US)

(72) Inventor: Wayne Rothbaum, Delray Beach, FL (US)

(73) Assignee: QUOGUE IP HOLDINGS LLC, New York, NY (US)

(21) Appl. No.: 17/776,836

(22) PCT Filed: Nov. 13, 2020
(86) PCT No.: PCT/US2020/060422

§ 371 Date: May 13, 2022

Related U.S. Application Data
(60) Provisional application No. 62/935,178, filed on Nov. 14, 2019.
(60) Provisional application No. 63/012,723, filed on Apr. 20, 2020.

(51) Int. Cl. A61K 31/519; A61P 35/00; A61K 31/451

(52) U.S. Cl. CPC A61K 31/519; A61P 35/00; A61K 31/451

(57) ABSTRACT

Therapeutic methods and pharmaceutical compositions for treating a cancer, including a B cell hematological malignancy selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

### FIELD OF THE INVENTION

Methods of treating a cancer using a combination of a Mouse double minute 2 homolog (MDM2) inhibitor and a BTK inhibitor.

### BACKGROUND OF THE INVENTION

p53 is a tumor suppressor and transcription factor that responds to cellular stress by activating the transcription of numerous genes involved in cell cycle arrest, apoptosis, senescence, and DNA repair. Unlike normal cells, which have infrequent cause for p53 activation, tumor cells are under constant cellular stress from various insults including hypoxia and pro-apoptotic oncogene activation. Thus, there is a strong selective advantage for inactivation of the p53 pathway in tumors, and it has been proposed that eliminating p53 function may be a prerequisite for tumor survival. In support of this notion, three groups of investigators have used mouse models to demonstrate that absence of p53 function is a continuous requirement for the maintenance of established tumors. When the investigators restored p53 function to tumors with inactivated p53, the tumors regressed.

p53 is inactivated by mutation and/or loss in 50% of solid tumors and 10% of liquid tumors. Other key members of the p53 pathway are also genetically or epigenetically altered in cancer. MDM2, an oncoprotein, inhibits p53 function, and it is activated by gene amplification at incidence rates that are reported to be as high as 10%. MDM2, in turn, is inhibited by another tumor suppressor, p14ARF. It has been suggested that alterations downstream of p53 may be responsible for at least partially inactivating the p53 pathway in p53 WT tumors (p53 wild type). In support of this concept, some p53WT tumors appear to exhibit reduced apoptotic capacity, although their capacity to undergo cell cycle arrest remains intact. One cancer treatment strategy involves the use of small molecules that bind MDM2 and neutralize its interaction with p53. MDM2 inhibits p53 activity by three mechanisms: 1) acting as an E3 ubiquitin ligase to promote p53 degradation; 2) binding to and blocking the p53 transcriptional activation domain; and 3) exporting p53 from the nucleus to the cytoplasm. All three of these mechanisms would be blocked by neutralizing the MDM2-p53 interaction. In particular, this therapeutic strategy could be applied to tumors that are p53 WT, and studies with small molecule MDM2 inhibitors have yielded promising reductions in tumor growth both in vitro and in vivo. Further, in patients with p53-inactivated tumors, stabilization of wild type p53 in normal tissues by MDM2 inhibition might allow selective protection of normal tissues from mitotic poisons. As used herein, MDM2 means a human MDM2 protein and p53 means a human p53 protein. It is noted that human MDM2 can also be referred to as HDM2 or hMDM2. Several MDM2 inhibitors are in human clinical trials for the treatment of various cancers.

Bruton's tyrosine kinase (BTK) is a Tec family non-receptor protein kinase, expressed in B cells and myeloid cells. The function of BTK in signaling pathways activated by the engagement of the B cell receptor (BCR) and FcεR1 on mast cells is well established. In addition, a function for BTK as a downstream target in Toll like receptor signaling was suggested. Functional mutations in BTK in humans results in the primary immunodeficiency disease called XLA which is characterized by a defect in B cell development with a block between pro- and pre-B cell stage. This results in an almost complete absence of B lymphocytes in human causing a pronounced reduction of serum immunoglobulin of all classes. These findings support the key role for BTK in the regulation of the production of auto-antibodies in autoimmune diseases. In addition, regulation of BTK may affect BCR-induced production of pro-inflammatory cytokines and chemokines by B cells, indicating a broad potential for BTK in the treatment of autoimmune diseases.

With the regulatory role reported for BTK in FcεR-mediated mast cell activation, BTK inhibitors may also show potential in the treatment of allergic responses (Gilfillan, Immunological Reviews, 2009, 288, 149-169).

Furthermore, BTK is also reported to be implicated in RANKL-induced osteoclast differentiation (Shinohara, Cell, 2008, 132, 794-806) and therefore may also be of interest for the treatment of bone resorption disorders.

Other diseases with an important role for dysfunctional B cells are B cell malignancies. Indeed anti-CD20 therapy is used effectively in the clinic for the treatment of follicular lymphoma, diffuse large B-cell lymphoma and chronic lymphocytic leukemia (Lim, Haematologica, 2010, 95, 135-143). The reported role for BTK in the regulation of proliferation and apoptosis of B cells indicates there is potential for BTK inhibitors in the treatment of B cell lymphomas as well. Inhibition of BTK seems to be relevant in particular for B cell lymphomas due to chronic active BCR signaling (Davis, Nature, 2010, 463, 88-92).

The present invention relates to methods of treating a cancer in a human subject with an MDM2 inhibitor and a BTK inhibitor.

### SUMMARY OF THE INVENTION

The present invention relates to a method of treating a cancer, comprising co-administering, to a human subject in need thereof, one or more compositions comprising therapeutically effective amount of (1) an MDM2 inhibitor or a pharmaceutically acceptable salt thereof, and (2) a Bruton's tyrosine kinase (BTK) inhibitor or a pharmaceutically acceptable salt thereof.

In an embodiment, the MDM2 inhibitor is administered before administration of the BTK inhibitor.

In an embodiment, the MDM2 inhibitor is administered concurrently with the administration of the BTK inhibitor.

In an embodiment, the MDM2 inhibitor is administered to the subject after administration of the BTK inhibitor.

In an embodiment, the MDM2 inhibitor is selected from the group consisting of the compounds listed in Table 1 or a pharmaceutically-acceptable salt thereof.

In an embodiment, the MDM2 inhibitor is a compound of Formula (I) or a compound of Formula (II):

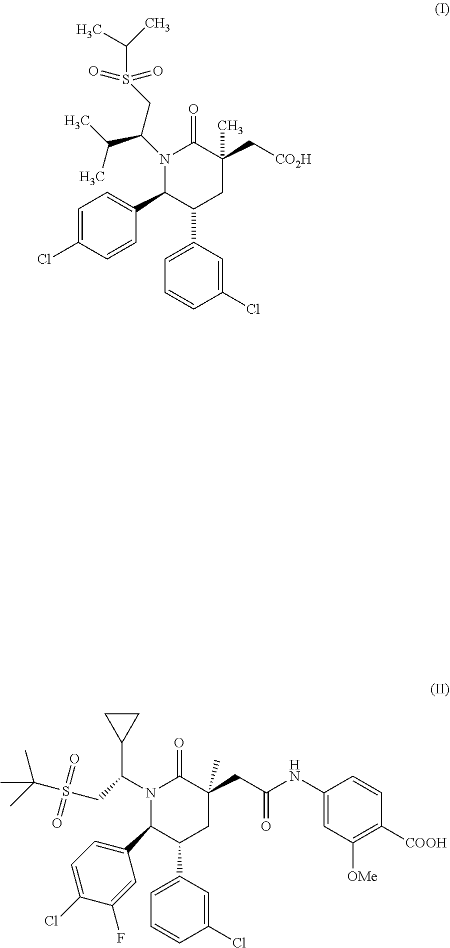 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

In an embodiment, the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the BTK inhibitor is selected from the group consisting of the compounds listed in Table 2 or pharmaceutically-acceptable salt thereof.

In an embodiment, the BTK inhibitor is a compound of Formula (III) (Ibrutinib) or a compound of Formula (IV) (Acalabrutinib):

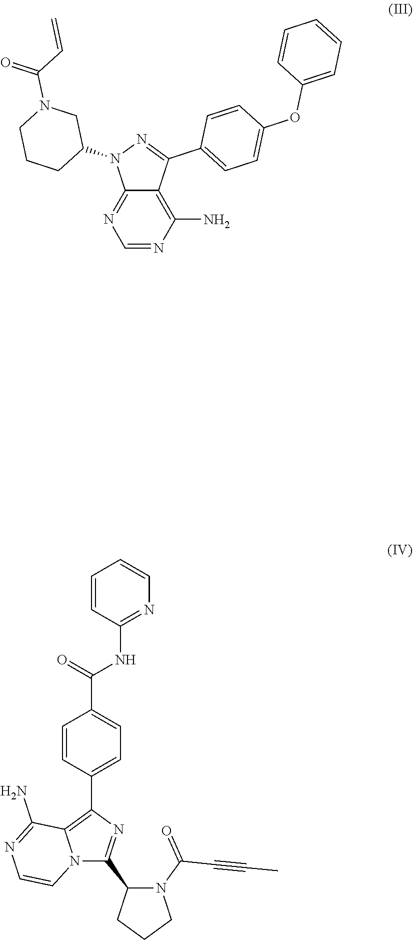

In an embodiment, the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the cancer is a B cell hematological malignancy.

In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

In an embodiment, the cancer is selected from the group consisting of myelofibrosis, multiple myeloma, and acute myeloid leukemia.

In another aspect, the present invention relates to a pharmaceutical composition comprising therapeutically effective amounts of an MDM2 inhibitor or a pharmaceutically acceptable salt thereof, and (2) a Bruton's tyrosine kinase (BTK) inhibitor or a pharmaceutically acceptable salt thereof, for use in the treatment of cancer.

In an embodiment, the MDM2 inhibitor is selected from the group consisting of the compounds listed in Table 1 or a pharmaceutically-acceptable salt thereof.

In an embodiment, the MDM2 inhibitor is a compound of Formula (I) or a compound of Formula (II):

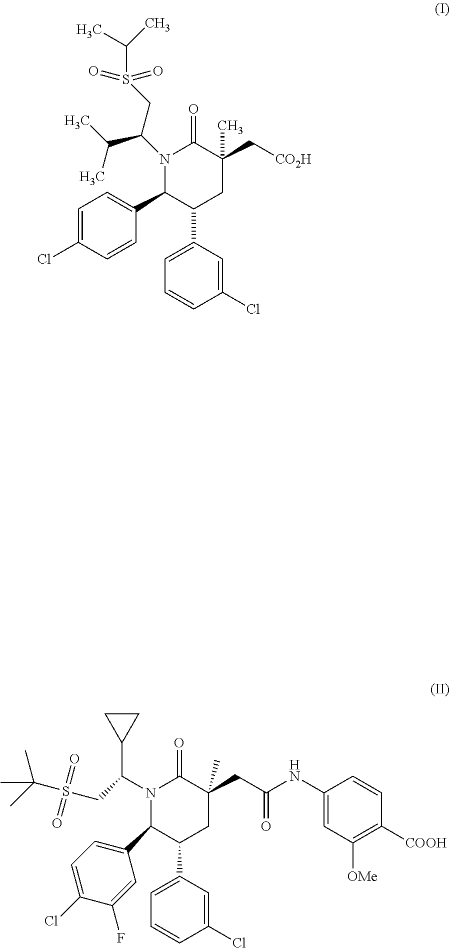 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

In an embodiment, the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the BTK inhibitor is selected from the group consisting of the compounds listed in Table 2 or pharmaceutically-acceptable salt thereof.

In an embodiment, the BTK inhibitor is a compound of Formula (III) (Ibrutinib) or a compound of Formula (IV) (Acalabrutinib):

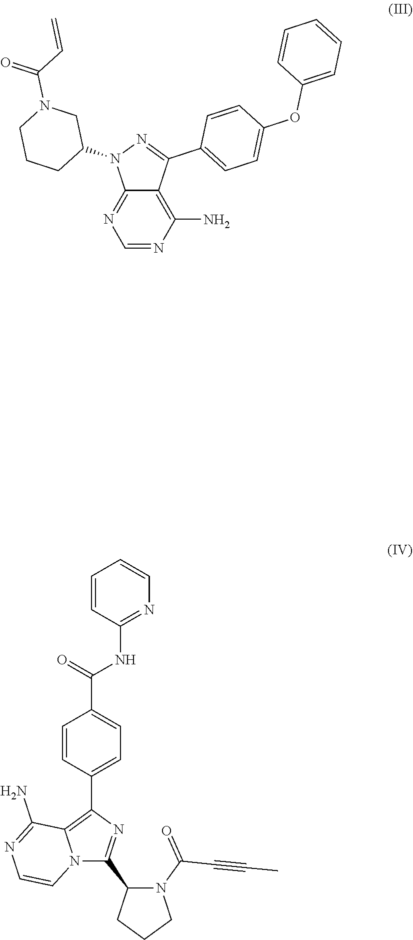

In an embodiment, the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the cancer is a B cell hematological malignancy.

In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

In an embodiment, the cancer is selected from the group consisting of myelofibrosis, multiple myeloma, and acute myeloid leukemia.

In another aspect, the present invention relates to a combination comprising a Bruton's tyrosine kinase (BTK) inhibitor and an MDM2 inhibitor, or pharmaceutically acceptable salts thereof.

In an embodiment, the combination is in the form of a pharmaceutical composition.

In an embodiment, the combination is in the form of a kit comprising two or more pharmaceutical compositions and optionally a package insert or label providing directions for administering the pharmaceutical compositions simultaneously, separately or sequentially, wherein the two or more pharmaceutical compositions together comprise an MDM2 inhibitor and a BTK inhibitor or pharmaceutically acceptable salts thereof.

In an embodiment, the MDM2 inhibitor is selected from the group consisting of the compounds listed in Table 1 or a pharmaceutically-acceptable salt thereof.

In an embodiment, the MDM2 inhibitor is a compound of Formula (I) or a compound of Formula (II):

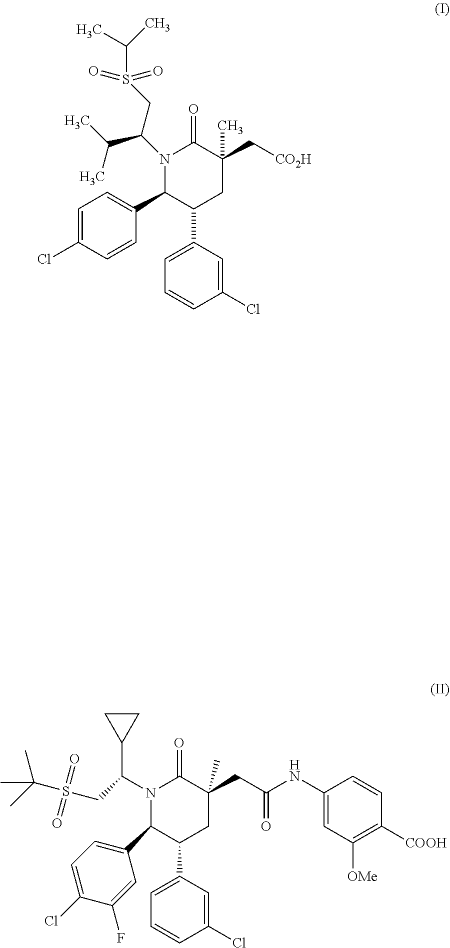 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

In an embodiment, the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the BTK inhibitor is selected from the group consisting of the compounds listed in Table 2 or pharmaceutically-acceptable salt thereof.

In an embodiment, the BTK inhibitor is a compound of Formula (III) (Ibrutinib) or a compound of Formula (IV) (Acalabrutinib):

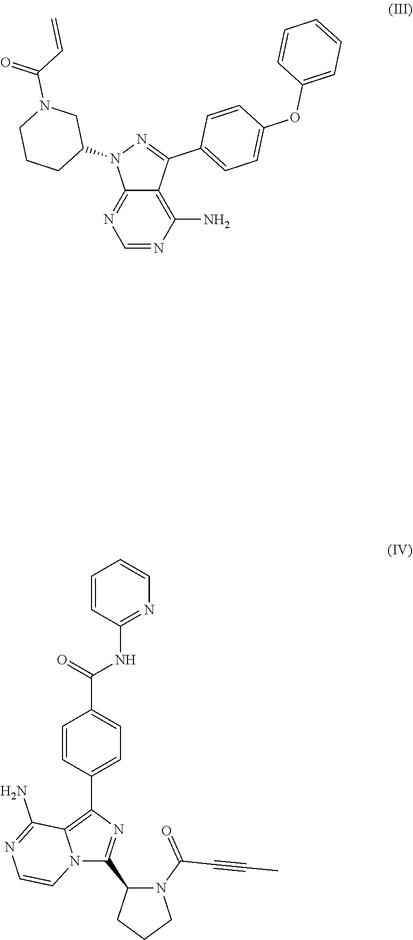

In an embodiment, the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the cancer is a B cell hematological malignancy.

In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

In an embodiment, the cancer is selected from the group consisting of myelofibrosis, multiple myeloma, and acute myeloid leukemia.

### BRIEF DESCRIPTION OF THE DRAWINGS

The foregoing summary, as well as the following detailed description of the invention, will be better understood when read in conjunction with the appended drawings.

FIG. 1 illustrates the changes of blasts in peripheral blood after treating the AML patients (N=16 Evaluable) with an MDM2 inhibitor—the compound of Formula (I).

FIG. 2 illustrates the changes of blasts in bone marrow after treating the AML patients (N=19 Evaluable) with an MDM2 inhibitor—the compound of Formula (I).

FIG. 3 illustrates mobilization of lymphocytes into the peripheral circulation after BTK inhibition. Ibr=Ibrutinib; Veh=Vehicle.

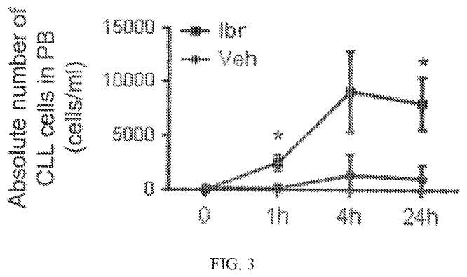

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

Unless defined otherwise, all technical and scientific terms used herein have the same meaning as is commonly understood by one of skill in the art to which this invention belongs.

The terms “administered in combination with” and “co-administration” as used herein, encompass administration of two or more active pharmaceutical ingredients to a subject so that both agents and/or their metabolites are present in the subject at the same time. Co-administration includes simultaneous administration in separate compositions, administration at different times in separate compositions, or administration in a composition in which two or more agents are present.

The term “combination” or “pharmaceutical combination” is defined herein to refer to either a fixed combination in one dosage unit form, a non-fixed combination or a kit of parts for the combined administration where the therapeutic agents may be administered together, independently at the same time or separately within time intervals, which preferably allows that the combination partners show a cooperative, e.g. synergistic effect. Thus, the single compounds of the pharmaceutical combination of the present disclosure could be administered simultaneously or sequentially.

Furthermore, the pharmaceutical combination of the present disclosure may be in the form of a fixed combination or in the form of a non-fixed combination.

The term “effective amount” or “therapeutically effective amount” refers to that amount of an active pharmaceutical ingredient or combination of active pharmaceutical ingredients as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells, (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The terms “enantiomerically enriched,” “enantiomerically pure,” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, such as at least 80% by weight. In some embodiments, the enrichment can be significantly greater than 80% by weight, providing a “substantially enantiomerically enriched,” “substantially enantiomerically pure,” or a “substantially non-racemic” preparation, which refers to preparations of compositions which have at least 85% by weight of one enantiomer relative to the other enantiomer, such as at least 90% by weight, and such as at least 95% by weight. The terms “diastereomerically enriched” and “diastereomerically pure,” as used herein, refer to compositions in which the percent by weight of one diastereomer is greater than the amount of that one diastereomer in a control mixture of diastereomers. In some embodiments, the enrichment can be significantly greater than 80% by weight, providing a “substantially diastereomerically enriched” or “substantially diastereomerically pure” preparation, which refers to preparations of compositions which have at least 85% by weight of one diastereomer relative to other diastereomers, such as at least 90% by weight, and such as at least 95% by weight.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (H PLC) and the formation and crystallization of chiral salts; or some enantiomers can be prepared by asymmetric syntheses. See, for example, Jacques, Enantiomers, Racemates and Resolutions, Wiley Interscience, New York (1981); E. L. Eliel and S. H. Wilen, Stereochemistry of Organic Compounds, Wiley-Interscience, New York (1994).

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)- and 20% (R)-, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

The term “fixed combination” means that the therapeutic agents, e.g., the single compounds of the combination, are in the form of a single entity or dosage form.

The term “IC₅₀” refers to the half maximal inhibitory concentration, i.e. inhibition of 50% of the desired activity. The term “EC₅₀” refers to the drug concentration at which one-half the maximum response is achieved.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

In an embodiment, compounds described herein include of the isomers, stereoisomers, and enantiomers thereof.

The term “non-fixed combination” means that the therapeutic agents, e.g., the single compounds of the combination, are administered to a patient as separate entities or dosage forms either simultaneously or sequentially with no specific time limits, wherein preferably such administration provides therapeutically effective levels of the two therapeutic agents in the body of the subject, e.g., a mammal or human in need thereof.

“Pharmaceutically acceptable carrier” or “pharmaceutically acceptable excipient” is intended to include any and all solvents, dispersion media, coatings, antibacterial and antifungal agents, isotonic, and absorption delaying agents. The use of such media and agents for active pharmaceutical ingredients is well known in the art. Except insofar as any conventional media or agent is incompatible with the active pharmaceutical ingredient, its use in the therapeutic compositions of the invention is contemplated. Supplementary active ingredients can also be incorporated into the described compositions. Unless otherwise specified, or clearly indicated by the text, reference to therapeutic agents useful in the pharmaceutical combination of the present disclosure includes both the free base of the compounds, and all pharmaceutically acceptable salts of the compounds.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve proton transfer between the cocrystal and the drug, and instead involves intermolecular interactions, such as hydrogen bonding, aromatic ring stacking, or dispersive forces, between the cocrystal former and the drug in the crystal structure.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

“Solvate” refers to a compound in physical association with one or more molecules of a pharmaceutically acceptable solvent.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Compounds of the invention also include crystalline and amorphous forms, including, for example, polymorphs, pseudopolymorphs, solvates, hydrates, unsolvated polymorphs (including anhydrates), conformational polymorphs, and amorphous forms of the compounds, as well as combinations thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as combinations thereof, unless a particular crystalline or amorphous form is referred to.

### Co-Administration of Compounds

Bruton's tyrosine kinase (BTK) is involved in the regulation of B-cell growth, migration and adhesion. The importance of BTK in cell trafficking is emphasized by the clonal contraction proceeded by lymphocytosis typical for the enzyme inhibitor, Ibrutinib, a BTK inhibitor, in B-cell malignancies, including chronic lymphocytic leukemia (CLL). Inhibiting BTK by Ibrutinib reduced surface membrane (sm) levels of CXCR4 but not CXCRS, CD49d and other adhesion/homing receptors. Decreased smCXCR4 levels resulted in rapid re-distribution of CLL cells from spleens and lymph nodes into the circulation. CLL cells with impaired smCXCR4 from BTK inhibition failed to home to spleens. These functional changes mainly resulted from inhibition of CXCR4 phosphorylation at Ser339, mediated directly by blocking BTK enzymatic activity and indirectly by affecting the function of downstream targets PLCγ2 and PKCμ, and eventually synthesis of PIM-1 and BTK itself. Chen, Leukemia 2016, 30, 833-843.

It was also found that the expression of CXCR4 is down-regulated on the CD34+ cells of patients with myelofibrosis with myeloid metaplasia. Rosti, Blood Cells, Molecules and Diseases, 2007, 38, 280-286. Blocking the CXCR4 receptor appears to be capable of “mobilizing” hematopoietic stem cells into the bloodstream as peripheral blood stem cells.

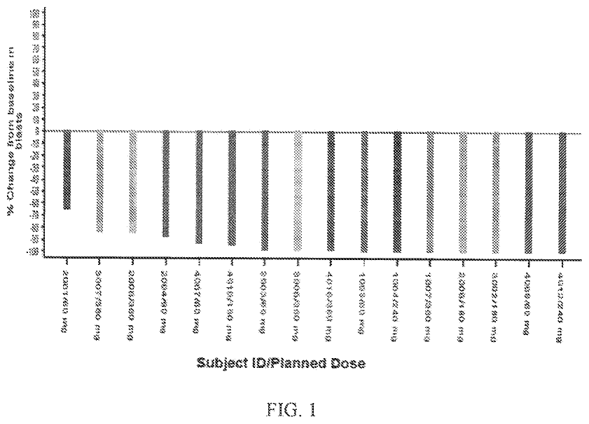

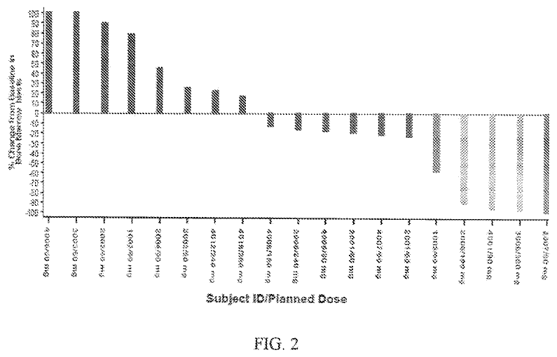

As shown in FIGS. 1-2 illustrating the test results of a clinical trial using an MDM2 inhibitor for the treatment AML, 100% of the blasts in the peripheral blood were wiped out, compared with a much lower percent in the bone marrow. Therefore, without wishing to be bound by theory, the combination of an MDM2 inhibitor and a BTK inhibitor would show synergistic effects for treating B-cell malignancies and myeloid diseases.

Therefore, the present invention relates to pharmaceutical combinations or pharmaceutical compositions that are particularly useful as a medicine. Specifically, the combinations or compositions of the present disclosure can be applied in the treatment of a cancer. In an embodiment, the cancer is a B cell hematological malignancy selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM). In an embodiment, the cancer is selected from the group consisting of myelofibrosis, multiple myeloma, and acute myeloid leukemia. In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD). The present invention also relates to use of pharmaceutical combinations or pharmaceutical compositions of the present disclosure for the preparation of a medicament for the treatment of a cancer, and to a method for treating a cancer in a subject in need thereof comprising administering to the subject a therapeutically effective amount of a pharmaceutical combination according to the present disclosure, or the pharmaceutical composition according to the present disclosure.

The present invention relates to a method of treating a cancer, comprising co-administering, to a human subject in need thereof, one or more compositions comprising therapeutically effective amount of (1) an MDM2 inhibitor or a pharmaceutically acceptable salt thereof, and (2) a Bruton's tyrosine kinase (BTK) inhibitor or a pharmaceutically acceptable salt thereof.

In an embodiment, the MDM2 inhibitor is administered before administration of the BTK inhibitor.

In an embodiment, the MDM2 inhibitor is administered concurrently with the administration of the BTK inhibitor.

In an embodiment, the MDM2 inhibitor is administered to the subject after administration of the BTK inhibitor.

In an embodiment, the MDM2 inhibitor is selected from the group consisting of the compounds listed in Table 1 or a pharmaceutically-acceptable salt thereof.

TABLE 1
MDM2 Inhibitors

| No. | IUPAC Name |
|---|---|
| 1. | 2-[(3R,5R,6S)-5-(3-chlorophenyl)-6-(4-chlorophenyl)-3-methyl-1-[(2S)-3-methyl-1-propan-2- |
| | ylsulfonylbutan-2-yl]-2-oxopiperidin-3-yl]acetic acid |
| 2. | 4-[[(2R,3S,4R,5S)-3-(3-chloro-2-fluorophenyl)-4-(4-chloro-2-fluorophenyl)-4-cyano-5-(2,2- |
| | dimethylpropyl)pyrrolidine-2-carbonyl]amino]-3-methoxybenzoic acid |
| 3. | (5bS,6aS,7aS,8R,8aR,9aS,9bS,10aS,10bS)-8-hydroxy-8a-isopropyl-10b-methyl- |
| | 2,5,5b,6,6a,8,8a,9a,9b,10b-decahydrotris(oxireno)[2′,3′:4b,5;2″,3″:6,7;2″′,3″′:8a,9]phenanthro[1,2- |
| | c]furan-3(1H)-one |
| 4. | 4-[(4S,5R)-4,5-bis(4-chlorophenyl)-2-(4-methoxy-2-propan-2-yloxyphenyl)-4,5- |
| | dihydroimidazole-1-carbonyl]piperazin-2-one |
| 5. | (4S)-5-(5-chloro-1-methyl-2-oxopyridin-3-yl)-4-(4-chlorophenyl)-2-(2,4-dimethoxypyrimidin- |
| | 5-yl)-3-propan-2-yl-4H-pyrrolo[3,4-d]imidazol-6-one |
| 6. | [(4S,5R)-2-(4-tert-butyl-2-ethoxyphenyl)-4,5-bis(4-chlorophenyl)-4,5-dimethylimidazol-1-yl]- |
| | [4-(3-methylsulfonylpropyl)piperazin-1-yl]methanone |
| 7. | (1S)-1-(4-chlorophenyl)-6-methoxy-2-[4-[methyl-[[4-(4-methyl-3-oxopiperazin-1- |
| | yl)cyclohexyl]methyl]amino]phenyl]-7-propan-2-yloxy-1,4-dihydroisoquinolin-3-one |
| 8. | 4-[4,5-bis(4-chlorophenyl)-2-(4-methoxy-2-propan-2-yloxyphenyl)-4,5-dihydroimidazole-1- |
| | carbonyl]piperazin-2-one |
| 9. | methyl 2-[2-chloro-6-ethoxy-4-[(3-methyl-5-oxo-1-phenylpyrazol-4- |
| | ylidene)methyl]phenoxy]acetate |
| 10. | (2′R,3R,3′S,5′S)-6-chloro-3′-(3-chloro-2-fluorophenyl)-5′-(2,2-dimethylpropyl)-N-(4- |
| | hydroxycyclohexyl)-2-oxospiro[1H-indole-3,4′-pyrrolidine]-2′-carboxamide |
| 11. | (2′R,3S,3′S,5′R)-6-chloro-3′-(3-chloro-2-fluorophenyl)-5′-(2,2-dimethylpropyl)-N-(4- |
| | hydroxycyclohexyl)-2-oxospiro[1H-indole-3,4′-pyrrolidine]-2′-carboxamide |
| 12. | 4-[8-[(3,4-dimethylphenyl)sulfamoyl]-3a,4,5,9b-tetrahydro-3H-cyclopenta[c]quinolin-4- |
| | yl]benzoic acid |
| 13. | ethyl 3-[2-(tert-butylamino)-1-[(4-chlorophenyl)methyl-formylamino]-2-oxoethyl]-6-chloro- |
| | 1H-indole-2-carboxylate |
| 14. | (2′R,3R,3′S,5′S)-N-(4-carbamoyl-2-methoxyphenyl)-6-chloro-3′-(3-chloro-2-fluorophenyl)-5′- |
| | (2,2-dimethylpropyl)-2-oxospiro[1H-indole-3,4′-pyrrolidine]-2′-carboxamide |
| 15. | 4-[(4R,5S)-4,5-bis(4-chlorophenyl)-2-(4-methoxy-2-propan-2-yloxyphenyl)-4,5- |
| | dihydroimidazole-1-carbonyl]piperazin-2-one |
| 16. | 1-N-[2-(1H-indol-3-yl)ethyl]-4-N-pyridin-4-ylbenzene-1,4-diamine |
| 17. | (E)-1-(4-methylpiperazin-1-yl)-3-(5-nitrofuran-2-yl)prop-2-en-1-one |
| 18. | 2-[4-[(4S,5R)-2-(4-tert-butyl-2-ethoxyphenyl)-4,5-bis(4-chlorophenyl)-4,5- |
| | dimethylimidazole-1-carbonyl]piperazin-1-yl]-1-morpholin-4-ylethanone |
| 19. | (1R)-1-(4-chlorophenyl)-6-methoxy-2-[4-[methyl-[[4-(4-methyl-3-oxopiperazin-1- |
| | yl)cyclohexyl]methyl]amino]phenyl]-7-propan-2-yloxy-1,4-dihydroisoquinolin-3-one |
| 20. | 4-amino-1-[(2R,3S,4S,5R)-3,4-dihydroxy-5-(hydroxymethyl)oxolan-2-yl]pyrimidin-2-one |
| 21. | (3′R,4′S,5′R)-N-((3R,6S)-6-carbamoyltetrahydro-2H-pyran-3-yl)-6″-chloro-4′-(2-chloro-3- |
| | fluoropyridin-4-yl)-4,4-dimethyl-2″-oxodispiro[cyclohexane-1,2′-pyrrolidine-3′,3″-indoline]- |
| | 5′-carboxamide |
| 22. | (3′R,4′S,5′R)-N-((3R,6S)-6-carbamoyltetrahydro-2H-pyran-3-yl)-6″-chloro-4′-(2-chloro-3- |
| | fluoropyridin-4-yl)-4,4-dimethyl-2″-oxodispiro[cyclohexane-1,2′-pyrrolidine-3′,3″-indoline]- |
| | 5′-carboxamide 4-methylbenzenesulfonate |
| 23. | 4-((3′R,4′S,5′R)-6″-Chloro-4′-(3-chloro-2-fluorophenyl)-1′-ethyl-2″-oxodispiro[cyclohexane- |
| | 1,2′-pyrrolidine-3′,3″-indoline]-5′-carboxamido)bicyclo[2.2.2]octane-1-carboxylic Acid |
| 24. | 4-((3′R,4′S,5′R)-6″-chloro-4′-(3-chloro-2-fluorophenyl)-2″-oxodispiro[cyclohexane-1,2′- |
| | pyrrolidine-3′,3″-indoline]-5′-carboxamido)benzoic acid |

In an embodiment, the MDM2 inhibitor is a compound of Formula (I) or a compound of Formula (II):

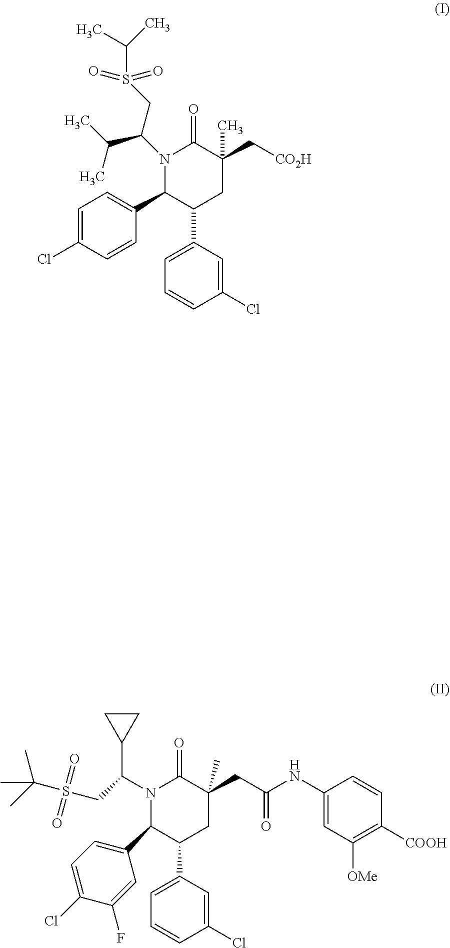 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

In an embodiment, the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the BTK inhibitor is selected from the group consisting of the compounds listed in Table 2 or pharmaceutically-acceptable salt thereof.

TABLE 2
BTK Inhibitors

| No. | IUPAC Name |
|---|---|
| 1. | Acalabrutinib ((S)-4-(8-amino-3-(1-(but-2-ynoyl)pyrrolidin-2-yl)imidazo[1,5-a]pyrazin-1- |
| | yl)-N-(pyridin-2-yl)benzamide) |
| 2. | Ibrutinib (1-[(3R)-3-[4-amino-3-(4-phenoxyphenyl)pyrazolo[3,4-d]pyrimidin-1- |
| | yl]piperidin-1-yl]prop-2-en-1-one) |
| 3. | (7S)-2-(4-phenoxyphenyl)-7-(1-prop-2-enoylpiperidin-4-yl)-4,5,6,7- |
| | tetrahydropyrazolo[1,5-a]pyrimidine-3-carboxamide |
| 4. | 2-(4-phenoxyphenyl)-7-(1-prop-2-enoylpiperidin-4-yl)-4,5,6,7-tetrahydropyrazolo[1,5- |
| | a]pyrimidine-3-carboxamide |
| 5. | 7(R)-2-(4-phenoxyphenyl)-7-(1-prop-2-enoylpiperidin-4-yl)-4,5,6,7- |
| | tetrahydropyrazolo[1,5-a]pyrimidine-3-carboxamide |
| 6. | 6-amino-9-[(3R)-1-but-2-ynoylpyrrolidin-3-yl]-7-(4-phenoxyphenyl)purin-8-one |
| 7. | N-[3-[[5-fluoro-2-[4-(2-methoxyethoxy)anilino]pyrimidin-4-yl]amino]phenyl]prop-2- |
| | enamide |
| 8. | Fenebrutinib (10-[3-(hydroxymethyl)-4-[1-methyl-5-[[5-[(2S)-2-methyl-4-(oxetan-3- |
| | yl)piperazin-1-yl]pyridin-2-yl]amino]-6-oxopyridin-3-yl]pyridin-2-yl]-4,4-dimethyl-1,10- |
| | diazatricyclo[6.4.0.0^{2,6}]dodeca-2(6),7-dien-9-one) |
| 9. | 1-[4-[[[6-amino-5-(4-phenoxyphenyl)pyrimidin-4-yl]amino]methyl]piperidin-1-yl]prop- |
| | 2-en-1-one |
| 10. | 1-[4-[[[6-amino-5-(4-phenoxyphenyl)pyrimidin-4-yl]amino]methyl]piperidin-1-yl]prop- |
| | 2-en-1-one |
| 11. | (2-chloro-4-phenoxyphenyl)-[4-[[(3R,6S)-6-(hydroxymethyl)oxan-3-yl]amino]-7H- |
| | pyrrolo[2,3-d]pyrimidin-5-yl]methanone |
| 12. | N-[3-[6-[4-[(2R)-1,4-dimethyl-3-oxopiperazin-2-yl]anilino]-4-methyl-5-oxopyrazin-2-yl]- |
| | 2-methylphenyl]-4,5,6,7-tetrahydro-1-benzothiophene-2-carboxamide |
| 13. | 2-[2-[2-[4-[4-amino-3-(4-phenoxyphenyl)pyrazolo[3,4-d]pyrimidin-1-yl]piperidin-1- |
| | yl]ethoxy]ethoxy]-N-[2-(2,6-dioxopiperidin-3-yl)-1,3-dioxoisoindol-5-yl]acetamide |
| 14. | N-[3-[2-[4-(4-methylpiperazin-1-yl)anilino]furo[3,2-d]pyrimidin-4-yl]oxyphenyl]prop-2- |
| | enamide |
| 15. | 4-tert-butyl-N-[2-methyl-3-[1-methyl-5-[4-(morpholine-4-carbonyl)-3-(prop-2- |
| | enoylamino)anilino]-6-oxopyridin-3-yl]phenyl]benzamide |
| 16. | (R,E)-2-(3-(4-amino-3-(2-fluoro-4-phenoxyphenyl)-1H-pyrazolo[3,4-d]pyrimidin-1- |
| | yl)piperidine-1-carbonyl)-4-methyl-4-(4-(oxetan-3-yl)piperazin-1-yl)pent-2-enenitrile |
| 17. | Branebrutinib ((S)-4-(3-(but-2-ynamido)piperidin-1-yl)-5-fluoro-2,3-dimethyl-1H- |
| | indole-7-carboxamide) |
| 18. | 4-(tert-Butyl)-N-(2-methyl-3-(4-methyl-6-((4-(morpholine-4-carbonyl)phenyl)amino)-5- |
| | oxo-4,5-dihydropyrazin-2-yl)phenyl)benzamide |
| 19. | N-(1-(7H-Pyrrolo[2,3-d]pyrimidin-4-yl)piperidin-3-yl)-2-((3-chlorophenyl)amino) |
| | acetamide |
| 20. | 6-cyclopropyl-8-fluoro-2-[2-(hydroxymethyl)-3-[1-methyl-5-[[5-(4-methylpiperazin-1- |
| | yl)pyridin-2-yl]amino]-6-oxopyridin-3-yl]phenyl]isoquinolin-1-one |
| 21. | N-[5-[9-[4-(methanesulfonamido)phenyl]-2-oxobenzo[h][1,6]naphthyridin-1-yl]-2- |
| | methylphenyl]prop-2-enamide |
| 22. | 4-(4-((4-((3-acrylamidophenyl)amino)-5-fluoropyrimidin-2-yl)amino)phenoxy)-N- |
| | methylpicolinamide |
| 23. | (7S)-3-fluoro-4-[3-(8-fluoro-1-methyl-2,4-dioxoquinazolin-3-yl)-2-methylphenyl]-7-(2- |
| | hydroxypropan-2-yl)-6,7,8,9-tetrahydro-5H-carbazole-1-carboxamide |
| 24. | 1-[3-fluoro-4-[7-(5-methyl-1H-imidazol-2-yl)-1-oxo-2,3-dihydroisoindol-4-yl]phenyl]-3- |
| | [3-(trifluoromethyl)phenyl]urea |
| 25. | 9-(1-methylpyrazol-4-yl)-1-(1-prop-2-enoyl-2,3-dihydroindol-6- |
| | yl)benzo[h][1,6]naphthyridin-2-one |
| 26. | 7-(2-hydroxypropan-2-yl)-4-[2-methyl-3-(4-oxoquinazolin-3-yl)phenyl]-9H-carbazole-1- |
| | carboxamide |
| 27. | 10-[2-(Hydroxymethyl)-3-[1-methyl-6-oxo-5-(pyrimidin-4-ylamino)pyridin-3-yl]phenyl]- |
| | 4,4-dimethyl-7-thia-10-azatricyclo[6.4.0.02,6]dodeca-1(8),2(6)-dien-9-one |
| 28. | (S)-5-amino-1-(1-cyanopiperidin-3-yl)-3-(4-(2,4-difluorophenoxy)phenyl)-1H-pyrazole- |
| | 4-carboxamide |
| 29. | (S)-4-(3-(1-Acryloylpyrrolidin-2-yl)-8-aminoimidazo[1,5-a]pyrazin-1-yl)-N-(pyridin-2- |
| | yl)benzamide |
| 30. | (S,E)-4-(8-Amino-3-(1-(4-(dimethylamino)but-2-enoyl)pyrrolidin-2-yl)imidazo[1.5- |
| | a]pyrazin-1-yl)-N(pyridin-2-yl)benzamide |
| 31. | (S)-4-(8-Amino-3-(1-but-2-ynoylpyrrolidin-2-yl)imidazo[1,5-a]pyrazin-1-yl)-N-(4- |
| | methylpyridin-2-yl)benzamide |
| 32. | (S,E)-4-(8-Amino-3-(1-(4-methoxybut-2-enoyl)pyrrolidin-2-yl)imidazo[1,5-a]pyrazin-1- |
| | yl)-N-(4-propylpyridin-2-yl)benzamide |
| 33. | (S)-4-(8-Amino-3-(1-but-2-ynoylpyrrolidin-2-yl)imidazo[1,5-a]pyrazin-1-yl)-N-(4- |
| | (trifluoromethyl)pyridin-2-yl)benzamide |
| 34. | (S)-4-(8-Amino-3-(1-but-2-ynoylpyrrolidin-2-yl)imidazo[1,5-a]pyrazin-1-yl)-N-(4,5,6,7- |
| | tetrahydrobenzo[d]thiazol-2-yl)benzamide |
| 35. | (S)-4-(3-(1-acryloylpyrrolidin-2-yl)-8-aminoimidazo[1,5-a]pyrazin-1-yl)-2-fluoro-N- |
| | (pyridin-2-yl)benzamide |
| 36. | (S)-4-(3-(1-Acryloylpyrrolidin-2-yl)-8-aminoimidazo[1,5-a]pyrazin-1-yl)-2-methoxy-N- |
| | (pyridin-2-yl)benzamide |
| 37. | (S,E)-4-(8-Amino-3-(1-(4-(dimethylamino)but-2-enoyl)pyrrolidin-2-yl)imidazo[1,5- |
| | a]pyrazin-1-yl)-N-(thiazol-2-yl)benzamide |
| 38. | (S)-4-(3-(1-Acryloylpiperidin-2-yl)-8-aminoimidazo[1,5-a]pyrazin-1-yl)-N-(4- |
| | fluoropyridin-2-yl)benzamide |
| 39. | (S)-4-(3-(1-Acryloylpiperidin-2-yl)-8-aminoimidazo[1,5-a]pyrazin-1-yl)-N-(4- |
| | cyanopyridin-2-yl)benzamide |
| 40. | (S)-4-(8-Amino-3-(1-(vinylsulfonyl)piperidin-2-yl)imidazo[1,5-a]pyrazin-1-yl)-N-(4- |
| | (trifluoromethyl)pyridin-2-yl)benzamide |
| 41. | (S)-4-(3-(1-Acryloylpiperidin-2-yl)-8-aminoimidazo[1,5-a]pyrazin-1-yl)-N-(pyrimidin-2- |
| | yl)benzamide |
| 42. | (S)-4-(3-(1-Acryloylpiperidin-2-yl)-8-aminoimidazo[1,5-a]pyrazin-1-yl)-N-(4- |
| | methylpyrimidin-2-yl)benzamide |
| 43. | (S)-4-(8-Amino-3-(1-but-2-ynoylpiperidin-2-yl)imidazo[1,5-a]pyrazin-1-yl)-N- |
| | (pyrimidin-4-yl)benzamide |
| 44. | (S)-4-(8-Amino-3-(1-but-2-ynoylpiperidin-2-yl)imidazo[1,5-a]pyrazin-1-yl)-N- |
| | (pyridazin-3-yl)benzamide |
| 45. | (S,E)-4-(8-Amino-3-(1-(4-methoxybut-2-enoyl)piperidin-2-yl)imidazo[1,5-a]pyrazin-1- |
| | yl)-N-(5-ethylthiazol-2-yl)benzamide |
| 46. | (S)-4-(3-(1-Acryloylpiperidin-2-yl)-8-aminoimidazo[1,5-a]pyrazin-1-yl)-2-fluoro-N-(4- |
| | propylpyridin-2-yl)benzamide |
| 47. | (S,E)-4-(8-Amino-3-(1-(4-(dimethylamino)but-2-enoyl)piperidin-2-yl)imidazo[1,5- |
| | a]pyrazin-1-yl)-2-methoxy-N-(4-propylpyridin-2-yl)benzamide |
| 48. | 4-(8-Amino-3-((S)-1-but-2-ynoylpiperidin-2-yl)imidazo[1,5-a]pyrazin-1-yl)-3-methyl-N- |
| | (pyridin-2-yl)benzamide |
| 49. | 4-(3-(Acrylamidomethyl)-8-aminoimidazo[1,5-a]pyrazin-1-yl)-N-(pyridin-2- |
| | yl)benzamide |
| 50. | (S)-4-(8-Amino-3-(1-but-2-ynamidoethyl)imidazo[1,5-a]pyrazin-1-yl)-N-(pyridin-2- |
| | yl)benzamide |
| 51. | (S)-S-2-(2-(8-Amino-1-(4-(pyridin-2-ylcarbamoyl)phenyl)imidazo[1,5-a]pyrazin-3- |
| | yl)pyrrolidin-1-yl)-2-oxoethylethanethioate |
| 52. | (S)-4-(8-Amino-3-(1-(4-hydroxy-4-methylpent-2-ynoyl)pyrrolidin-2-yl)imidazo[1,5- |
| | a]pyrazin-1-yl)-N(pyridin-2-yl)benzamide |
| 53. | (S)-4-(8-Amino-3-(1-(6-chloropyrimidine-4-carbonyl)pyrrolidin-2-yl)imidazo[1,5- |
| | a]pyrazin-1-yl)-N-(pyridin-2-yl)benzamide |
| 54. | (S)-4-(8-Amino-3-(1-pent-2-ynoylpyrrolidin-2-yl)imidazo[1,5-a]pyrazin-1-yl)-N- |
| | (pyridin-2-yl)benzamide |
| 55. | (S)-4-(8-Amino-3-(1-(3-cyclopropylpropioloyl)pyrrolidin-2-yl)imidazo[1,5-a]pyrazin-1- |
| | yl)-N-(pyridin-2-yl)benzamide |
| 56. | (S)-4-(8-Amino-3-(1-hex-2-ynoylpyrrolidin-2-yl)imidazo[1,5-a]pyrazin-1-yl)-N-(pyridin- |
| | 2-yl)benzamide |
| 57. | 4-(3-(1-Acryloylazepan-2-yl)-8-aminoimidazo[1,5-a]pyrazin-1-yl)-N-(pyridin-2- |
| | yl)benzamide |
| 58. | (R)-4-(8-Amino-3-(4-but-2-ynoylmorpholin-3-yl)imidazo[1,5-a]pyrazin-1-yl)-N-(pyridin- |
| | 2-yl)benzamide |
| 59. | (S)-4-(8-amino-3-(1-(N-methylbut-2-ynamido)ethyl)imidazo[1,5-a]pyrazin-1-yl)-N-(4- |
| | (trifluoromethyl)pyridin-2-yl)benzamide |
| 60. | (S)-4-(3-(1-acryloylpyrrolidin-2-yl)-8-aminoimidazo[1,5-a]pyrazin-1-yl)-N-(4- |
| | fluoropyridin-2-yl)benzamide |
| 61. | (S)-4-(3-(1-acryloylpyrrolidin-2-yl)-8-aminoimidazo[1,5-a]pyrazin-1-yl)-N-(4-(pyrrolidin- |
| | 1-yl)pyridin-2-yl)benzamide |
| 62. | (S)-4-(8-amino-3-(1-but-2-ynoylpiperidin-2-yl)imidazo[1,5-a]pyrazin-1-yl)-N-(4- |
| | fluoropyridin-2-yl)benzamide |
| 63. | (S)-4-(8-amino-3-(1-but-2-ynoylpiperidin-2-yl)imidazo[1,5-a]pyrazin-1-yl)-N-(pyridine- |
| | 2-yl)benzamide |
| 64. | (S)-4-(3-(1-acryloylpiperidin-2-yl)-8-aminoimidazo[1,5-a]pyrazin-1-yl)-N-(pyridine-2- |
| | yl)benzamide |
| 65. | (S)-4-(8-amino-3-(1-but-2-ynoylpyrrolidin-2-yl)imidazo[1,5-a]pyrazin-1-yl)-N-(4- |
| | propylpyridin-2-yl)benzamide |
| 66. | (S,E)-4-(8-amino-3-(1-(4-methoxy-N-methylbut-2-enamido)ethyl)imidazo[1,5- |
| | a]pyrazin-1-yl)-N-(4-propylpyridin-2-yl)benzamide |
| 67. | (S)-4-(8-amino-3-(1-(vinylsulfonyl)piperidin-2-yl)imidazo[1,5-a]pyrazin-1-yi)-N-(4- |
| | propylpyridin-2-yl)benzamide |
| 68. | (S)-4-(3-(1-acryloylpiperidin-2-yl)-8-aminoimidazo[1,5-a]pyrazin-1-yl)-N-(4- |
| | propylpyridin-2-yl)benzamide |
| 69. | (S)-4-(3-(1-acryloylpyrrolidin-2-yl)-8-aminoimidazo[1,5-a]pyrazin-1-yl)-N-(4- |
| | (trifluoromethyl)pyridin-2-yl)benzamide |
| 70. | (S)-4-(8-amino-3-(1-but-2-ynoylpiperidin-2-yl)imidazo[1,5-a]pyrazin-1-yl)-N-(4- |
| | (trifluoromethyl)pyridin-2-yl)benzamide |
| 71. | (S)-4-(8-amino-3-(1-but-2-ynoylpiperidin-2-yl)imidazo[1,5-a]pyrazin-1-yl)-N-(4- |
| | propylpyridin-2-yl)benzamide |
| 72. | (S,E)-4-(8-amino-3-(1-(4-(dimethylamino)but-2-enoyl)pyrrolidin-2-yl)imidazo[1,5- |
| | a]pyrazin-1-yl)-N-(4-isopropylpyridin-2-yl)benzamide |
| 73. | 4-(8-amino-3-((S)-1-(vinylsulfonyl)piperidin-2-yl)imidazo[1,5-a]pyrazin-1-yl)-3-methyl- |
| | N-(pyridin-2-yl)benzamide |
| 74. | (S)-4-(8-amino-3-(1-but-2-ynoylpiperidin-2-yl)imidazo[1,5-a]pyrazin-1-yl)-2-fluoro-N- |
| | (4-propylpyridin-2-yl)benzamide |
| 75. | (S,E)-4-(8-amino-3-(1-(4-methoxy-N-methylbut-2-enamido)ethyl)imidazo[1,5- |
| | a]pyrazin-1-yl)-N-(4-(trifluoromethyl)pyridin-2-yl)benzamide |
| 76. | (S,E)-4-(8-amino-3-(1-(4-(dimethylamino)-N-methylbut-2-enamido)ethyl)imidazo[1,5- |
| | a]pyrazin-1-yl)-N-(4-propylpyridin-2-yl)benzamide |
| 77. | (S,E)-4-(8-amino-3-(1-(4-(pyrrolidin-1-yl)but-2-enoyl)pyrrolidin-2-yl)imidazo[1,5- |
| | a]pyrazin-1-yl)-N-(4-propylpyridin-2-yl)benzamide |
| 78. | (S,E)-4-(8-amino-3-(1-(4-(dimethylamino)but-2-enoyl)piperidin-2-yl)imidazo[1,5- |
| | a]pyrazin-1-yl)-N-(pyridin-2-yl)benzamide |
| 79. | (S)-4-(8-amino-3-(1-(2-chloropyrimidine-4-carbonyl)pyrrolidin-2-yl)imidazo[1,5- |
| | a]pyrazin-1-yl)-N-(4-propylpyridin-2-yl)benzamide |
| 80. | (S)-4-(3-(1-acrylamidoethyl)-8-aminoimidazo[1,5-a]pyrazin-1-yl)-N-(pyridin-2- |
| | yl)benzamide |
| 81. | (S)-4-(3-(1-acryloylpyrrolidin-2-yl)-8-aminoimidazo[1,5-a]pyrazin-1-yl)-N-(thiazol-2- |
| | yl)benzamide |
| 82. | (S)-4-(8-amino-3-(1-but-2-ynoylpyrrolidin-2-yl)imidazo[1,5-a]pyrazin-1-yl)-N-(4- |
| | isopropylpyridin-2-yl)benzamide |
| 83. | (S)-4-(8-amino-3-(1-(2-chloropyrimidine-4-carbonyl)piperidin-2-yl)imidazo[1,5- |
| | a]pyrazin-1-yl)-N-(4-propylpyridin-2-yl)benzamide |
| 84. | (S,E)-4-(8-amino-3-(1-(4-methoxybut-2-enoyl)piperidin-2-yl)imidazo[1,5-a]pyrazin-1- |
| | yl)-N-(4-(trifluoromethyl)pyridin-2-yl)benzamide |
| 85. | (S)-4-(3-(1-acryloylpiperidin-2-yl)-8-aminoimidazo[1,5-a]pyrazin-1-yl)-N-(4- |
| | (trifluoromethyl)pyridin-2-yl)benzamide |
| 86. | (S)-4-(8-amino-3-(1-but-2-ynoylpiperidin-2-yl)imidazo[1,5-a]pyrazin-1-yl)-2-methoxy- |
| | N-(4-propylpyridin-2-yl)benzamide |
| 87. | (S,E)-4-(8-amino-3-(1-(4-methoxybut-2-enoyl)piperidin-2-yl)imidazo[1,5-a]pyrazin-1- |
| | yl)-2-methoxy-N-(4-propylpyridin-2-yl)benzamide |
| 88. | (S)-4-(8-amino-3-(1-(2-chloropyrimidine-4-carbonyl)piperidin-2-yl)imidazo[1,5- |
| | a]pyrazin-1-yl)-N-(4-(trifluoromethyl)pyridin-2-yl)benzamide |
| 89. | (S)-4-(8-amino-3-(1-but-2-ynoylpiperidin-2-yl)imidazo[1,5-a]pyrazin-1-yi)-N-(5- |
| | ethylthiazol-2-yl)benzamide |
| 90. | (S)-4-(3-(1-acryloylpiperidin-2-yl)-8-aminoimidazo[1,5-a]pyrazin-1-yl)-N-(5- |
| | ethylthiazol-2-yl)benzamide |
| 91. | (S)-4-(8-amino-3-(1-(2-chloropyrimidine-4-carbonyl)piperidin-2-yl)imidazo[1,5- |
| | a]pyrazin-1-yl)-N-(5-ethylthiazol-2-yl)benzamide |
| 92. | (R,E)-4-(8-amino-3-(4-(4-methoxybut-2-enoyl)morpholin-3-yl)imidazo[1,5-a]pyrazin- |
| | 1-yl)-N-(pyridin-2-yl)benzamide |
| 93. | (S,E)-4-(8-amino-3-(1-(4-methoxybut-2-enoyl)piperidin-2-yl)imidazo[1,5-a]pyrazin-1- |
| | yl)-N-(4-propylpyridin-2-yl)benzamide |
| 94. | (S)-4-(3-(1-acryloylpyrrolidin-2-yl)-8-aminoimidazo[1,5-a]pyrazin-1-yl)-N-(4- |
| | cyanopyridin-2-yl)benzamide |
| 95. | (S)-4-(8-amino-3-(1-but-2-ynoylpyrrolidin-2-yl)imidazo[1,5-a]pyrazin-1-yl)-N-(4- |
| | methoxypyridin-2-yl)benzamide |
| 96. | (S)-4-(3-(1-acryloylpyrrolidin-2-yl)-8-aminoimidazo[1,5-a]pyrazin-1-yl)-N-(4- |
| | methylpyridin-2-yl)benzamide |
| 97. | (S)-4-(3-(1-acryloylpyrrolidin-2-yl)-8-aminoimidazo[1,5-a]pyrazin-1-yl)-N-(4- |
| | propylpyridin-2-yl)benzamide |
| 98. | (S)-4-(3-(1-acryloylpyrrolidin-2-yl)-8-aminoimidazo[1,5-a]pyrazin-1-yl)-N-(4- |
| | ethylpyridin-2-yl)benzamide |
| 99. | (S,E)-4-(8-amino-3-(1-(4-(dimethylamino)but-2-enoyl)pyrrolidin-2-yl)imidazo[1,5- |
| | a]pyrazin-1-yl)-N-(pyridin-2-yl)benzamide |
| 100. | (S,E)-4-(8-amino-3-(1-(4-methoxybut-2-enoyl)pyrrolidin-2-yl)imidazo[1,5-a]pyrazin-1- |
| | yl)-N-(4-(trifluoromethyl)pyridin-2-yl)benzamide |
| 101. | (S)-4-(8-amino-3-(1-(2-chloropyrimidine-4-carbonyl)pyrrolidin-2-yl)imidazo[1,5- |
| | a]pyrazin-1-yl)-N-(4-methylpyridin-2-yl)benzamide |
| 102. | (S)-4-(8-amino-3-(1-but-2-ynoylpyrrolidin-2-yl)imidazo[1,5-a]pyrazin-1-yl)-N-(4- |
| | cyanopyridin-2-yl)benzamide |
| 103. | (S)-4-(8-amino-3-(1-but-2-ynoylpyrrolidin-2-yl)imidazo[1,5-a]pyrazin-1-yl)-N-(4- |
| | ethylpyridin-2-yl)benzamide |
| 104. | (S)-4-(8-amino-3-(1-but-2-ynoylpyrrolidin-2-yl)imidazo[1,5-a]pyrazin-1-yl)-N-(4- |
| | phenylpyridin-2-yl)benzamide |
| 105. | (S)-4-(3-(1-acryloylpyrrolidin-2-yl)-8-aminoimidazo[1,5-a]pyrazin-1-yi)N-(4- |
| | phenylpyridin-2-yl)benzamide |
| 106. | (R,E)-1-(3-(4-amino-3-(4-phenoxyphenyl)-1H-pyrazolo[3,4-d]pyrimidin-1-yl)piperidin-1- |
| | yl)-4-(dimethylamino)but-2-en-1-one |
| 107. | (E)-1-(3-(4-amino-3-(4-phenoxyphenyl)-1H-pyrazolo[3,4-d]pyrimidin-l-yl)piperidin-l-yl)- |
| | 4-morpholinobut-2-en-1-one |
| 108. | 1-(4-(4-amino-3-(4-phenoxyphenyl)-1H-pyrazolo[3,4-d]pyrimidin-1-yl)piperidin-1- |
| | yl)prop-2-en-1-one |
| 109. | (E)-1-(4-(4-amino-3-(4-phenoxyphenyl)-1H-pyrazolo[3,4-d]pyrimidin-l-yl)piperidin-l- |
| | yl)-4-(dimethylamino)but-2-en-l-one |
| 110. | (E)-N-((ls,4s)-4-(4-amino-3-(4-phenoxyphenyl)-1H-pyrazolo[3,4-d]pyrimidin-l- |
| | yl)cyclohexyl)-4-(dimethylamino)but-2-enamide |
| 111. | 1-(4-(4-amino-3-(4-phenoxyphenyl)-1H-pyrazolo[3,4-d]pyrimidin-1-yl)piperidin-1- |
| | yl)prop-2-en-1-one |
| 112. | N-((lr,4r)-4-(4-amino-3-(4-phenoxyphenyl)-1H-pyrazolo[3,4-d]pyrimidin-l- |
| | yl)cyclohexyl)acrylamide |
| 113. | (E)-1-((R)-2-((4-amino-3-(4-phenoxyphenyl)-1H-pyrazolo[3,4-d]pyrimidin-l- |
| | yl)methyl)pyrolidin-l-yl)-4-(dimethylamino)but-2-en-l-one |
| 114. | (E)-1-((S)-2-((4-amino-3-(4-phenoxyphenyl)-1H-pyrazolo[3,4-d]pyrimidin-l- |
| | yl)methyl)pyrolidin-l-yl)-4-(dimethylamino)but-2-en-l-one |
| 115. | l-((R)-2-((4-amino-3-(4-phenoxyphenyl)-1H-pyrazolo[3,4-d]pyrimidin-l- |
| | yl)methyl)pyrrolidin-l-yl)prop-2-en-l-one |
| 116. | l-((S)-2-((4-amino-3-(4-phenoxyphenyl)-1H-pyrazolo[3,4-d]pyrimidin-l- |
| | yl)methyl)pyrrolidin-l-yl)prop-2-en-l-one |
| 117. | 1((R)-2-((4-amino-3-(4-phenoxyphenyl)-1H-pyrazolo[3,4-d]pyrimidin-l- |
| | yl)methyl)pyrrolidin-l-yl)but-2-yn-lone |
| 118. | 1-((S)-2-((4-amino-3-(4-phenoxyphenyl)-1H-pyrazolo[3,4-d]pyrimidin-l- |
| | yl)methyl)pyrrolidin-l-yl)but-2-yn-l-one |
| 119. | l-((R)-3-(4-amino-3-(4-phenoxyphenyl)-1H-pyrazolo[3,4-d]pyrimidin-l-yl)piperidin-l- |
| | yl)but-2-yn-l-one |
| 120. | (E)-N-((lr,4r)-4-(4-amino-3-(4-phenoxyphenyl)-1H-pyrazolo[3,4-d]pyrimidin-l- |
| | yl)cyclohexyl-4-(dimethylamino)but-2-enamide |
| 121. | N-(2-(4-amino-3-(4-phenoxyphenyl)-1H-pyrazolo[3,4-d]pyrimidin-l-yl)ethyl)-N- |
| | methylacrylamide |
| 122. | (E)-1-(4-(4-amino-3-(4-phenoxyphenyl)-1H-pyrazolo[3,4-d]pyrimidin-l-yl)-4- |
| | morpholinobut-2-en-1-one |
| 123. | (E)-1-((S-2-((4-amino-3-(4-phenoxyphenyl)-1H-pyrazolo[3,4-d]pyrimidin-lyl) |
| | methyl)pyrrolidin-l-yl)-4-morpholinobut-2-en-l-one |
| 124. | N-((ls,4s)-4-(4-amino-3-(4-phenoxyphenyl)-1H-pyrazolo[3,4-d]pyrimidin-l- |
| | yl)cyclohexyl)but-2-ynamide |
| 125. | N-(2-(4-amino-3-(4-phenoxyphenyl)-1H-pyrazolo[3,4-d]pyrimidin-l-yl)ethyl)acrylamide |
| 126. | (E)-1-((R)-3-(4-amino-3-(4-phenoxyphenyl)-1H-pyrazolo[3,4-d]pyrimidin-l-yl)piperidin- |
| | l-yl)-4-morpholinobut-2-en-l-one |
| 127. | (E)-N-((ls,4s)-4-(4-amino-3-(4-phenoxyphenyl)-1H-pyrazolo[3,4-d]pyrimidin-l- |
| | yl)cyclohexyl)-4-morpholinobut-2-enamide |
| 128. | 1-(4-(((6-amino-5-(4-phenoxyphenyl)pyrimidin-4-yl)amino)methyl)-4-fluoropiperidin-1- |
| | yl)prop-2-en-1-one |
| 129. | N-[3-[[5-fluoro-2-[4-(2-methoxyethoxy)anilino]pyrimidin-4-yl]amino]phenyl]prop-2- |
| | enamid |
| 130. | 6-amino-9-[(3R)-1-but-2-ynoylpyrrolidin-3-yl]-7-(4-phenoxyphenyl)purin-8-one |
| 131. | (7S)-2-(4-phenoxyphenyl)-7-(1-prop-2-enoylpiperidin-4-yl)-4,5,6,7- |
| | tetrahydropyrazolo[1,5-a]pyrimidine-3-carboxamide |
| 132. | Orelabrutinib (2-(4-phenoxyphenyl)-6-(1-prop-2-enoylpiperidin-4-yl)pyridine-3- |
| | carboxamide) |
| 133. | Remibrutinib (N-[3-[6-amino-5-[2-[methyl(prop-2-enoyl)amino]ethoxy]pyrimidin-4-yl]- |
| | 5-fluoro-2-methylphenyl]-4-cyclopropyl-2-fluorobenzamide) |

In an embodiment, the BTK inhibitor is a compound of Formula (III) (Ibrutinib) or a compound of Formula (IV) (Acalabrutinib):

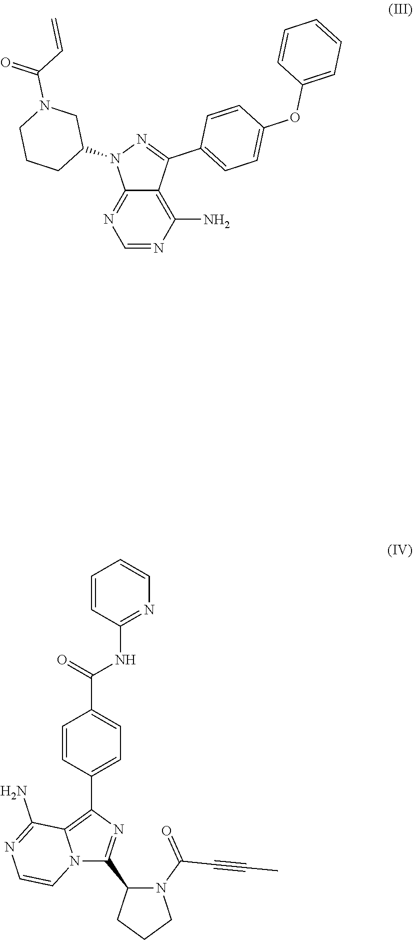

In an embodiment, the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the cancer is a B cell hematological malignancy.

In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

In an embodiment, the cancer is selected from the group consisting of myelofibrosis, multiple myeloma, and acute myeloid leukemia.

In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD).

In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF).

In an embodiment, the primary myelofibrosis (PMF) is selected from the group consisting of prefibrotic/early stage PMF and overt fibrotic stage PMF.

In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T).

In an embodiment, the present invention relates to a method of treating a cancer, comprising co-administering, to a human subject in need thereof, one or more compositions comprising therapeutically effective amount of (1) an MDM2 inhibitor or a pharmaceutically acceptable salt thereof, and (2) a Bruton's tyrosine kinase (BTK) inhibitor or a pharmaceutically acceptable salt thereof, wherein the MDM2 inhibitor is the compound of Formula (I) and the BTK inhibitor is Ibrutinib. In an embodiment, the compound of Formula (I) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (I) or a pharmaceutically acceptable salt thereof is administered on days 1 through 7 of a 28-day administration cycle. In an embodiment, Ibrutinib or a pharmaceutically acceptable salt thereof is administered once daily at a dose selected from the group consisting of 70 mg, 140 mg, 210 mg, 280 mg, 350 mg, 420 mg, 490 mg, and 560 mg. In an embodiment, the cancer is a B cell hematological malignancy. In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM). In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD). In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF). In an embodiment, the primary myelofibrosis (PM F) is selected from the group consisting of prefibrotic/early stage PMF and overt fibrotic stage PMF. In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T). In an embodiment, the cancer is selected from the group consisting of myelofibrosis, multiple myeloma, and acute myeloid leukemia.

In an embodiment, the present invention relates to a method of treating a cancer, comprising co-administering, to a human subject in need thereof, one or more compositions comprising therapeutically effective amount of (1) an MDM2 inhibitor or a pharmaceutically acceptable salt thereof, and (2) a Bruton's tyrosine kinase (BTK) inhibitor or a pharmaceutically acceptable salt thereof, wherein the MDM2 inhibitor is the compound of Formula (I) and the BTK inhibitor is Acalabrutinib. In an embodiment, the compound of Formula (I) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (I) or a pharmaceutically acceptable salt thereof is administered on days 1 through 7 of a 28-day administration cycle. In an embodiment, Acalabrutinib or a pharmaceutically acceptable salt thereof is administered twice daily at a dose selected from the group consisting of 50 mg, 100 mg, 150 mg, and 200 mg. In an embodiment, the cancer is a B cell hematological malignancy. In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM). In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD). In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF). In an embodiment, the primary myelofibrosis (PMF) is selected from the group consisting of prefibrotic/early stage PMF and overt fibrotic stage PMF. In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T). In an embodiment, the cancer is selected from the group consisting of myelofibrosis, multiple myeloma, and acute myeloid leukemia.

In an embodiment, the present invention relates to a method of treating a cancer, comprising co-administering, to a human subject in need thereof, one or more compositions comprising therapeutically effective amount of (1) an MDM2 inhibitor or a pharmaceutically acceptable salt thereof, and (2) a Bruton's tyrosine kinase (BTK) inhibitor or a pharmaceutically acceptable salt thereof, wherein the MDM2 inhibitor is the compound of Formula (II) and the BTK inhibitor is Ibrutinib. In an embodiment, the compound of Formula (II) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (II) or a pharmaceutically acceptable salt thereof is administered on days 1 through 7 of a 28-day administration cycle. In an embodiment, Ibrutinib or a pharmaceutically acceptable salt thereof is administered once daily at a dose selected from the group consisting of 70 mg, 140 mg, 210 mg, 280 mg, 350 mg, 420 mg, 490 mg, and 560 mg. In an embodiment, the cancer is a B cell hematological malignancy. In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM). In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD). In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF). In an embodiment, the primary myelofibrosis (PMF) is selected from the group consisting of prefibrotic/early stage PMF and overt fibrotic stage PMF. In an embodiment, the MPN is selected from the group consisting of chronic neutrophilic leukemia (CNL), chronic eosinophilic leukemia, chronic myelomonocytic leukemia (CM ML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T). In an embodiment, the cancer is selected from the group consisting of myelofibrosis, multiple myeloma, and acute myeloid leukemia.

In an embodiment, the present invention relates to a method of treating a cancer, comprising co-administering, to a human subject in need thereof, one or more compositions comprising therapeutically effective amount of (1) an MDM2 inhibitor or a pharmaceutically acceptable salt thereof, and (2) a Bruton's tyrosine kinase (BTK) inhibitor or a pharmaceutically acceptable salt thereof, wherein the MDM2 inhibitor is the compound of Formula (II) and the BTK inhibitor is Acalabrutinib. In an embodiment, the compound of Formula (II) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (II) or a pharmaceutically acceptable salt thereof is administered on days 1 through 7 of a 28-day administration cycle. In an embodiment, Acalabrutinib or a pharmaceutically acceptable salt thereof is administered twice daily at a dose selected from the group consisting of 50 mg, 100 mg, 150 mg, and 200 mg. In an embodiment, the cancer is a B cell hematological malignancy. In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM). In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD). In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF). In an embodiment, the primary myelofibrosis (PMF) is selected from the group consisting of prefibrotic/early stage PMF and overt fibrotic stage PMF. In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T). In an embodiment, the cancer is selected from the group consisting of myelofibrosis, multiple myeloma, and acute myeloid leukemia.

In another aspect, the present invention relates to a pharmaceutical composition comprising therapeutically effective amounts of an MDM2 inhibitor or a pharmaceutically acceptable salt thereof, and (2) a Bruton's tyrosine kinase (BTK) inhibitor or a pharmaceutically acceptable salt thereof, for use in the treatment of cancer.

In an embodiment, the MDM2 inhibitor is selected from the group consisting of the compounds listed in Table 1 or a pharmaceutically-acceptable salt thereof.

In an embodiment, the MDM2 inhibitor is a compound of Formula (I) or a compound of Formula (II):

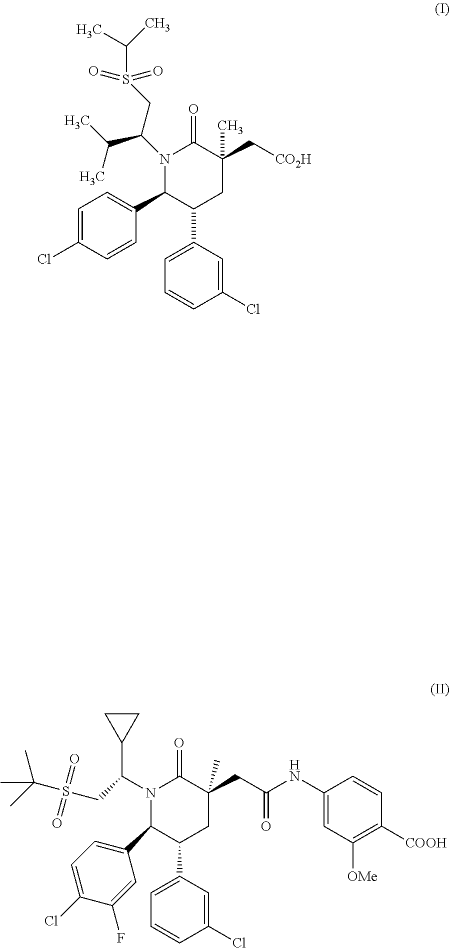 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

In an embodiment, the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the BTK inhibitor is selected from the group consisting of the compounds listed in Table 2 or pharmaceutically-acceptable salt thereof.

In an embodiment, the BTK inhibitor is a compound of Formula (III) (Ibrutinib) or a compound of Formula (IV) (Acalabrutinib).

In an embodiment, the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the cancer is a B cell hematological malignancy.

In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

In an embodiment, the cancer is selected from the group consisting of myelofibrosis, multiple myeloma, and acute myeloid leukemia.

In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD).

In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF).

In an embodiment, the primary myelofibrosis (PMF) is selected from the group consisting of prefibrotic/early stage PMF and overt fibrotic stage PMF.

In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T).

In an embodiment, the present invention relates to a pharmaceutical composition comprising therapeutically effective amounts of an MDM2 inhibitor or a pharmaceutically acceptable salt thereof, and (2) a Bruton's tyrosine kinase (BTK) inhibitor or a pharmaceutically acceptable salt thereof, for use in the treatment of cancer, wherein the MDM2 inhibitor is the compound of Formula (I) and the BTK inhibitor is Ibrutinib. In an embodiment, the compound of Formula (I) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (I) or a pharmaceutically acceptable salt thereof is administered on days 1 through 7 of a 28-day administration cycle. In an embodiment, Ibrutinib or a pharmaceutically acceptable salt thereof is administered once daily at a dose selected from the group consisting of 70 mg, 140 mg, 210 mg, 280 mg, 350 mg, 420 mg, 490 mg, and 560 mg.

In an embodiment, the present invention relates to a pharmaceutical composition comprising therapeutically effective amounts of an MDM2 inhibitor or a pharmaceutically acceptable salt thereof, and (2) a Bruton's tyrosine kinase (BTK) inhibitor or a pharmaceutically acceptable salt thereof, for use in the treatment of cancer, wherein the MDM2 inhibitor is the compound of Formula (I) and the BTK inhibitor is Acalabrutinib. In an embodiment, the compound of Formula (I) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (I) or a pharmaceutically acceptable salt thereof is administered on days 1 through 7 of a 28-day administration cycle. In an embodiment, Acalabrutinib or a pharmaceutically acceptable salt thereof is administered twice daily at a dose selected from the group consisting of 50 mg, 100 mg, 150 mg, and 200 mg.

In an embodiment, the present invention relates to a pharmaceutical composition comprising therapeutically effective amounts of an MDM2 inhibitor or a pharmaceutically acceptable salt thereof, and (2) a Bruton's tyrosine kinase (BTK) inhibitor or a pharmaceutically acceptable salt thereof, for use in the treatment of cancer, wherein the MDM2 inhibitor is the compound of Formula (II) and the BTK inhibitor is Ibrutinib. In an embodiment, the compound of Formula (II) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (II) or a pharmaceutically acceptable salt thereof is administered on days 1 through 7 of a 28-day administration cycle. In an embodiment, Ibrutinib or a pharmaceutically acceptable salt thereof is administered once daily at a dose selected from the group consisting of 70 mg, 140 mg, 210 mg, 280 mg, 350 mg, 420 mg, 490 mg, and 560 mg.

In an embodiment, the present invention relates to a pharmaceutical composition comprising therapeutically effective amounts of an MDM2 inhibitor or a pharmaceutically acceptable salt thereof, and (2) a Bruton's tyrosine kinase (BTK) inhibitor or a pharmaceutically acceptable salt thereof, for use in the treatment of cancer, wherein the MDM2 inhibitor is the compound of Formula (II) and the BTK inhibitor is Acalabrutinib. In an embodiment, the compound of Formula (II) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (II) or a pharmaceutically acceptable salt thereof is administered on days 1 through 7 of a 28-day administration cycle. In an embodiment, Acalabrutinib or a pharmaceutically acceptable salt thereof is administered twice daily at a dose selected from the group consisting of 50 mg, 100 mg, 150 mg, and 200 mg.

In another aspect, the present invention relates to a combination comprising a Bruton's tyrosine kinase (BTK) inhibitor and an MDM2 inhibitor, or pharmaceutically acceptable salts thereof.

In an embodiment, the combination is in the form of a pharmaceutical composition.

In an embodiment, the combination is in the form of a kit comprising two or more pharmaceutical compositions and optionally a package insert or label providing directions for administering the pharmaceutical compositions simultaneously, separately or sequentially, wherein the two or more pharmaceutical compositions together comprise an MDM2 inhibitor and a BTK inhibitor or pharmaceutically acceptable salts thereof.

In an embodiment, the MDM2 inhibitor is selected from the group consisting of the compounds listed in Table 1 or a pharmaceutically-acceptable salt thereof.

In an embodiment, the MDM2 inhibitor is a compound of Formula (I) or a compound of Formula (II).

In an embodiment, the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the BTK inhibitor is selected from the group consisting of the compounds listed in Table 2 or pharmaceutically-acceptable salt thereof.

In an embodiment, the BTK inhibitor is a compound of Formula (III) (Ibrutinib) or a compound of Formula (IV) (Acalabrutinib).

In an embodiment, the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

In an embodiment, the cancer is a B cell hematological malignancy.

In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

In an embodiment, the cancer is selected from the group consisting of myelofibrosis, multiple myeloma, and acute myeloid leukemia.

In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD).

In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF).

In an embodiment, the primary myelofibrosis (PMF) is selected from the group consisting of prefibrotic/early stage PMF and overt fibrotic stage PMF.

In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T).

In an embodiment, the present invention relates to a combination comprising a Bruton's tyrosine kinase (BTK) inhibitor and an MDM2 inhibitor, or pharmaceutically acceptable salts thereof, wherein the MDM2 inhibitor is the compound of Formula (I) and the BTK inhibitor is Ibrutinib. In an embodiment, the compound of Formula (I) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (I) or a pharmaceutically acceptable salt thereof is administered on days 1 through 7 of a 28-day administration cycle. In an embodiment, Ibrutinib or a pharmaceutically acceptable salt thereof is administered once daily at a dose selected from the group consisting of 70 mg, 140 mg, 210 mg, 280 mg, 350 mg, 420 mg, 490 mg, and 560 mg.

In an embodiment, the present invention relates to a combination comprising a Bruton's tyrosine kinase (BTK) inhibitor and an MDM2 inhibitor, or pharmaceutically acceptable salts thereof, wherein the MDM2 inhibitor is the compound of Formula (I) and the BTK inhibitor is Acalabrutinib. In an embodiment, the compound of Formula (I) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (I) or a pharmaceutically acceptable salt thereof is administered on days 1 through 7 of a 28-day administration cycle. In an embodiment, Acalabrutinib or a pharmaceutically acceptable salt thereof is administered twice daily at a dose selected from the group consisting of 50 mg, 100 mg, 150 mg, and 200 mg.

In an embodiment, the present invention relates to a combination comprising a Bruton's tyrosine kinase (BTK) inhibitor and an MDM2 inhibitor, or pharmaceutically acceptable salts thereof, wherein the MDM2 inhibitor is the compound of Formula (II) and the BTK inhibitor is Ibrutinib. In an embodiment, the compound of Formula (II) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (II) or a pharmaceutically acceptable salt thereof is administered on days 1 through 7 of a 28-day administration cycle. In an embodiment, Ibrutinib or a pharmaceutically acceptable salt thereof is administered once daily at a dose selected from the group consisting of 70 mg, 140 mg, 210 mg, 280 mg, 350 mg, 420 mg, 490 mg, and 560 mg.

In an embodiment, the present invention relates to a combination comprising a Bruton's tyrosine kinase (BTK) inhibitor and an MDM2 inhibitor, or pharmaceutically acceptable salts thereof, wherein the MDM2 inhibitor is the compound of Formula (II) and the BTK inhibitor is Acalabrutinib. In an embodiment, the compound of Formula (II) or a pharmaceutically acceptable salt thereof is administered once daily at a dose selected from the group consisting of 60 mg, 120 mg, 180 mg, 240 mg, 300 mg, 360 mg, 420 mg, and 480 mg. In an embodiment, the compound of Formula (II) or a pharmaceutically acceptable salt thereof is administered on days 1 through 7 of a 28-day administration cycle. In an embodiment, Acalabrutinib or a pharmaceutically acceptable salt thereof is administered twice daily at a dose selected from the group consisting of 50 mg, 100 mg, 150 mg, and 200 mg.

### Pharmaceutical Compositions for Oral Administration

In selected embodiments, the invention provides a pharmaceutical composition for oral administration a combination comprising an MDM2 inhibitor and a BTK inhibitor.

In selected embodiments, the invention provides a solid pharmaceutical composition for oral administration containing: (i) a combination comprising an effective amount of an MDM2 inhibitor and a BTK inhibitor, in combination with (ii) a pharmaceutical excipient suitable for oral administration. In selected embodiments, the composition further contains (iii) an effective amount of at least one additional active ingredient.

In selected embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

A combination of an MDM2 inhibitor and a BTK inhibitor can be further combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and combinations thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and combinations thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or combinations thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or combinations thereof. Additional lubricants include, for example, a syloid silica gel, a coagulated aerosol of synthetic silica, or combinations thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and combinations thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and combinations thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and combinations thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and combinations thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and combinations thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and combinations thereof. Within this group, preferred lipophilic surfactants include glycerol fatty acid esters, propylene glycol fatty acid esters, and combinations thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present invention and to minimize precipitation of the compound of the present invention. This can be especially important for compositions for non-oral use, such as for compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and combinations thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Examples may include, but are not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In selected embodiments, the invention provides a pharmaceutical composition for injection comprising a combination comprising an MDM2 inhibitor and a BTK inhibitor, and a pharmaceutical excipient suitable for injection.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable combinations thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an MDM2 inhibitor and a BTK inhibitor in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Administration of a combination comprising an MDM2 inhibitor and a BTK inhibitor can be effected by any method that enables delivery of the compounds to the site of action. These methods include oral routes, intraduodenal routes, parenteral injection (including intravenous, intra-arterial, subcutaneous, intramuscular, intravascular, or infusion), topical (e.g., transdermal application), via local delivery by catheter or stent.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include an MDM2 inhibitor and a BTK inhibitor, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer. In an embodiment, the invention provides a kit comprising a combination comprising an MDM2 inhibitor and a BTK inhibitor for use in the treatment of a cancer. In an embodiment, the cancer is a B cell hematological malignancy. In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM). In an embodiment, the cancer is a myeloproliferative neoplasm (MPN) selected from the group consisting of polycythemia vera (PV), myelofibrosis, primary myelofibrosis, thrombocythemia, essential thrombocythemia (ET), idiopathic myelofibrosis, systemic mastocystosis (SM), chronic neutrophilic leukemia (CNL), myelodysplastic syndrome (MDS), and systemic mast cell disease (SMCD). In an embodiment, the cancer is myelofibrosis selected from the group consisting of primary myelofibrosis (PMF), post-polycythemia vera myelofibrosis (post-PV MF), and post-essential thrombocythemia myelofibrosis (post-ET MF). In an embodiment, the primary myelofibrosis (PMF) is selected from the group consisting of prefibrotic/early stage PMF and overt fibrotic stage PMF. In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T). In an embodiment, the cancer is selected from the group consisting of myelofibrosis, multiple myeloma, and acute myeloid leukemia.

### Dosages and Dosing Regimens

The amount of an MDM2 inhibitor and a BTK inhibitor administered will be independently dependent on the human being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In some embodiments, an MDM2 inhibitor and a BTK inhibitor are independently administered in a single dose. Typically, such administration will be oral or by injection - e.g., intravenous injection, in order to introduce the agents quickly. However, other routes may be used as appropriate. A single dose of an MDM2 inhibitor and a BTK inhibitor may also be used for treatment of an acute condition.

In an embodiment, dosing may be once, twice, three times, four times, five times, six times, or more than six times per day. In an embodiment, dosing may be selected from the group consisting of once a day, twice a day, three times a day, four times a day, five times a day, six times a day, once every other day, once weekly, twice weekly, three times weekly, four times weekly, biweekly, and monthly. In some embodiments an MDM2 inhibitor and a BTK inhibitor are independently administered three times a week, including every Monday, Wednesday, and Friday.

Administration of an MDM2 inhibitor and a BTK inhibitor may independently continue as long as necessary. In some embodiments, the MDM2 inhibitor and the BTK inhibitor are independently administered for more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. In some embodiments, the MDM2 inhibitor and the BTK inhibitor are independently administered for less than 28, 14, 7, 6, 5, 4, 3, 2, or 1 day. In some embodiments, the MDM2 inhibitor and the BTK inhibitor are independently administered for about 14 days, about 21 days, about 28 days, about 35 days, about 42 days, about 49 days, or about 56 days. In some embodiments, the MDM2 inhibitor and the BTK inhibitor are independently administered chronically on an ongoing basis—e.g., for the treatment of chronic effects. In another embodiment, the administration of the MDM2 inhibitor and the BTK inhibitor independently continues for less than about 7 days. In yet another embodiment the administration continues for more than about 6, 10, 14, 28 days, two months, three months, four months, five months, six months, seven months, eight months, nine months, ten months, eleven months or one year. In some embodiments, the administration continues for more than about one year, two years, three years, four years, or five years. In some embodiments, continuous dosing is achieved and maintained as long as necessary.

In some embodiments, an effective dosage of the MDM2 inhibitor and the BTK inhibitor is independently in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of the MDM2 inhibitor or the BTK inhibitor is independently about 25 mg, about 50 mg, about 60 mg, about 70 mg, about 75 mg, about 100 mg, about 120 mg, about 125 mg, about 140 mg, about 150 mg, about 175 mg, about 180 mg, about 200 mg, about 210 mg, about 225 mg, about 240 mg, about 250 mg, about 275 mg, about 280 mg, about 300 mg, about 325 mg, about 350 mg, about 360 mg, about 375 mg, about 400 mg, about 420 mg, about 425 mg, about 450 mg, about 475 mg, about 480 mg, about 490 mg, about 500 mg, about 540 mg, about 560 mg, about 600 mg, about 630 mg, or about 700 mg.

In some embodiments, an effective dosage of the MDM2 inhibitor or the BTK inhibitor is independently in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an MDM2 inhibitor or a pharmaceutically acceptable salt thereof is administered at a dosage of 10 to 700 mg BID, including a dosage of 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 540 mg, 560 mg, 600 mg, 630 mg, or 700 mg BID.

In some embodiments, an MDM2 inhibitor or a pharmaceutically acceptable salt thereof is administered at a dosage of 10 to 700 mg QD, including a dosage of 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 540 mg, 560 mg, 600 mg, 630 mg, or 700 mg QD.

In some embodiments, a BTK inhibitor or a pharmaceutically acceptable salt thereof is administered at a dosage of 10 to 700 mg BID, including a dosage of 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 540 mg, 560 mg, 600 mg, 630 mg, or 700 mg BID.

In some embodiments, a BTK inhibitor or a pharmaceutically acceptable salt thereof is administered at a dosage of 10 to 700 mg QD, including a dosage of 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 540 mg, 560 mg, 600 mg, 630 mg, or 700 mg QD.

An effective amount of the MDM2 inhibitor or the BTK inhibitor may be administered in either single or multiple doses by any of the accepted modes of administration of agents having similar utilities, including buccal, sublingual, and transdermal routes, by intra-arterial injection, intravenously, parenterally, intramuscularly, subcutaneously or orally.

In some embodiments, the MDM2 inhibitor or the BTK inhibitor are independently administered to a subject intermittently, known as intermittent administration. By “intermittent administration”, it is meant a period of administration of a therapeutically effective dose of an MDM2 inhibitor and/or the BTK inhibitor, followed by a time period of discontinuance, which is then followed by another administration period and so on. In each administration period, the dosing frequency can be independently select from three times daily, twice daily, daily, once weekly, twice weekly, three times weekly, four times weekly, five times weekly, six times weekly or monthly. In an embodiment, the MDM2 inhibitor is the compound of Formula (I) or Formula (II). In an embodiment, the MDM2 inhibitor is selected from the compounds listed in Table 1. In an embodiment, the BTK inhibitor is the compound of Formula (III) or Formula (IV). In an embodiment, the BTK inhibitor is selected from the compounds listed in Table 2.

By “period of discontinuance” or “discontinuance period” or “rest period”, it is meant to the length of time when discontinuing of the administration of the MDM2 inhibitor and/or the BTK inhibitor. The time period of discontinuance may be longer or shorter than the administration period or the same as the administration period. For example, where the administration period comprises three times daily, twice daily, daily, once weekly, twice weekly, three times weekly, four times weekly, five times weekly, six times weekly or monthly dosing, the discontinuance period may be at least about 2, 3, 4, 5, 6, 7, 8, 9, 10, 11, 12, 13, 14, 15, 16, 17, 18, 19, 20, 21, 22, 23, 24, 25, 26, 27, 28, 29, one month, two months, three months, four months or more days. During the discontinuance period, other therapeutic agents other than an MDM2 inhibitor and the BTK inhibitor may be administered.

In an embodiment, the MDM2 inhibitor is administered to a human intermittently; while the BTK inhibitor is administered to a human non-intermittently. In an embodiment, the BTK inhibitor is administered to a human intermittently; while the MDM2 inhibitor is administered to a human non-intermittently. In an embodiment, both the MDM2 inhibitor and the BTK inhibitor are administered to a human intermittently. In an embodiment, both the MDM2 inhibitor and the BTK inhibitor are administered to a human non-intermittently.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Combined Use of the Compound of Formula (I) and Ibrutinib

ABC-type DLBCL cell line TMD-8 cells is suspended to 1×10⁸cells/mL using phosphate-buffered saline. 0.1 mL of the prepared cell suspension is subcutaneously transplanted to each NOD-SCID mouse (female, 6 weeks old). On 6 days after the tumor inoculation, after confirmation that the average tumor volume exceeded 100 mm³, the mice are grouped (6 mice per group) on the basis of their tumor volume values. 25 mg/kg or 50 mg/kg the compound of Formula (I) or 100 mg/kg or 200 mg/kg Ibrutinib is orally administered by forced administration to the mice. For a combined use group, 25 mg/kg or 50 mg/kg the compound of Formula (I) and 100 mg/kg or 200 mg/kg Ibrutinib are orally administered sequentially by forced administration. The administration is performed once a day for 5 consecutive days (18 to 22 days after the tumor inoculation) from the date of grouping (18 days after the tumor inoculation), and after a 2-day drug holiday, performed once a day for 4 consecutive days (25 to 28 days after the tumor inoculation). The major axis (mm) and minor axis (mm) of tumor are measured over time using an electronic digital caliper. Tumor growth inhibition % (TGI %) on the date of assessment (29 days after the tumor inoculation) would be calculated according to calculation equation shown below for evaluation. Also, the body weights are measured over time using an automatic balance for small animals, and body weight change % is calculated according to calculation equation shown below to study the influence of drug administration on the body weights. In addition, the results of the last body weight measurement are used in dose calculation.

TGI (%)=(1−A/B)×100

A: Average tumor volume of the compound-administered group on the date of assessment (*)

B: Average tumor volume of the untreated control group on the date of assessment (*)

*: The tumor volume is calculated according to 1/2×[Major axis of tumor]×[Minor axis of tumor]×[Minor axis of tumor].

Body weight change (%)=Average body weight change % of the individuals (5)

Body weight change % of each individual=(1−BWn/BWs)×100

BWn: Body weight on day n

BWs: Body weight on the start day of administration

1. A method of treating a cancer, comprising co-administering, to a human subject in need thereof, one or more compositions comprising therapeutically effective amount of (1) an MDM2 inhibitor or a pharmaceutically acceptable salt thereof, and (2) a Bruton's tyrosine kinase (BTK) inhibitor or a pharmaceutically acceptable salt thereof.

2. The method of claim 1, wherein the MDM2 inhibitor is administered before administration of the BTK inhibitor.

3. The method of claim 1, wherein the MDM2 inhibitor is administered concurrently with the administration of the BTK inhibitor.

4. The method of claim 1, wherein the MDM2 inhibitor is administered to the subject after administration of the BTK inhibitor.

5. The method of claim 1, wherein the MDM2 inhibitor is selected from the group consisting of the compounds listed in Table 1 or a pharmaceutically-acceptable salt thereof.

6. The method of claim 5, wherein the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

7. The method of claim 1, wherein the BTK inhibitor is selected from the group consisting of the compounds listed in Table 2 or pharmaceutically-acceptable salt thereof.

8. The method of claim 7, wherein the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

9. The method of claim 1, wherein the cancer is a B cell hematological malignancy.

10. The method of claim 9, wherein the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

11. The method of claim 1, wherein the cancer is selected from the group consisting of myelofibrosis, multiple myeloma, and acute myeloid leukemia.

12. A pharmaceutical composition comprising therapeutically effective amounts of an MDM2 inhibitor or a pharmaceutically acceptable salt thereof, and (2) a Bruton's tyrosine kinase (BTK) inhibitor or a pharmaceutically acceptable salt thereof.

13. The pharmaceutical composition of claim 12, wherein the MDM2 inhibitor is any one of the compounds selected from Table 1 or a pharmaceutically-acceptable salt thereof.

14. The pharmaceutical composition of claim 13, wherein the therapeutically effective amount of the MDM2 inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

15. The pharmaceutical composition of claim 12, wherein the BTK inhibitor is any one of the compounds selected from Table 2 or a pharmaceutically-acceptable salt thereof.

16. The pharmaceutical composition of claim 15, wherein the therapeutically effective amount of the BTK inhibitor is selected from the group consisting of 5 mg, 10 mg, 12.5 mg, 15 mg, 20 mg, 25 mg, 50 mg, 60 mg, 70 mg, 75 mg, 100 mg, 120 mg, 125 mg, 140 mg, 150 mg, 175 mg, 180 mg, 200 mg, 210 mg, 225 mg, 240 mg, 250 mg, 275 mg, 280 mg, 300 mg, 325 mg, 350 mg, 360 mg, 375 mg, 400 mg, 420 mg, 425 mg, 450 mg, 475 mg, 480 mg, 490 mg, 500 mg, 525 mg, 540 mg, 550 mg, 560 mg, 600 mg, 630 mg, and 700 mg.

17-30. (canceled)
